(19) United States
(12) Patent Application Publication
(10) Pub. No.: US 2025/0099701 A1
(43) Pub. Date: Mar. 27, 2025

(54) RAPID EMERGENCY TOAD POISONING KIT AND METHODS OF USE THEREOF

(71) Applicant: TOAD BUSTERS, LLC, Port St. Lucie, FL (US)

(72) Inventors: Jeannine Tilford, Port St. Lucie, FL (US); Gary Rezeppa, Naples, FL (US)

(21) Appl. No.: 18/885,975

(22) Filed: Sep. 16, 2024

Related U.S. Application Data
(60) Provisional application No. 63/584,741, filed on Sep. 22, 2023.

(51) Int. Cl. A61M 16/00; A61K 33/44

(52) U.S. Cl. CPC A61M 16/0048; A61K 33/44; A61M 16/0087

(57) ABSTRACT

Systems and devices preferably provided as a kit with multiple components, and methods of use, for responding to toxic cane toad poisoning in animals.

### CROSS REFERENCE TO RELATED APPLICATIONS

In accordance with 37 C.F.R. 1.76, a claim of priority is included in an Application Data Sheet filed concurrently herewith. Accordingly, the present invention claims priority to U.S. Provisional Patent Application No. 63/584,741, entitled, “RAPID EMERGENCY TOAD POISONING KIT AND METHODS OF USE THEREOF”, filed on 22, 2023. The contents of the above referenced application are incorporated herein by reference in its entirety.

### FIELD OF THE INVENTION

The present invention relates to systems, devices, kits, and methods of using the systems, devices, and kits in treating toad poisoning; and more particularly, to systems, devices, kits, and methods of using the systems, devices, and kits for responding to toxic cane toad poisoning in animals.

### BACKGROUND OF THE INVENTION

Cane toads, also known as “bufo” or “marine” toads (Rhinella marina, formally known as Bufo marinus) are large, non-native amphibians which are poisonous to many animals, particularly household pets, such as dogs or cats. Cane toads house a poisonous white substance, bufotoxin, in large glands on the side of their head. When threatened, the cane toad releases bufotoxin to ward off the attacking predator. Often the “attacking predator” is a curious dog that bites or simply licks the toad.

Exposure to bufotoxin, either through the bite or lick, or being sprayed into their eyes, can have serous ramifications. Penetration of the bufotoxin into a dog's mucous membrane (inside of the mouth, the conjunctiva of the eye, lining of the stomach) may cause hypersalivation vocalization, bright red gums, vommiting, disorientation, stumbling, circling, seizures, hyperthermia, rapid, abnormal heartbeat, and coma. In the most severe cases, death can occur. Progression of the symptoms and poisonous effects are rapid once exposed to the toxin. As such, early treatment is critical to successful outcomes.

Once exposed to bufotoxin, many pet owners rush their dogs to a treatment site, such as their veterinarian's office or pet hospital for treatment. Such action, while critical for a successful outcome, requires travel time, not to mention the time it takes to get the animal into a car. As early treatment is critical to success, the time it takes to get to a treatment site can be an issue. While some dog owners try to administer their own self help, i.e., wash the dog's mouth out to remove the toxin, exposure to the toxin may not always be in a convenient place, i.e., at home or where a water source is available. The time it takes to locate the water source and apply it to the animal could be long, influencing the final outcome, i.e., resulting in poor outcomes.

The present invention provides one or more components, provided as a kit, which helps in treating an animal exposed to cane toad poisoning.

### SUMMARY OF THE INVENTION

Systems and devices preferably provided as a kit with multiple components, and methods of use, for responding to toxic cane toad poisoning in animals.

Accordingly, it is an objective of the invention to provide multiple components for use in treating an animal exposed to toxic cane toad poisoning.

It is a further objective of the invention to provide a kit for use in treating an animal exposed to toxic cane toad poisoning.

It is yet another objective of the invention to provide a method for use in treating an animal exposed to toxic cane toad poisoning.

It is a still further objective of the invention to provide a kit designed for rapid treatment of an animal exposed to toxic cane toad poisoning.

It is a further objective of the invention to provide a method designed for rapid treatment of an animal exposed to toxic cane toad poisoning.

It is yet another objective of the invention to provide a kit having the necessary components in a single location for use in treating an animal exposed to toxic cane toad poisoning.

It is a still further objective of the invention to provide a method having the necessary components in a single location for use in treating an animal exposed to toxic cane toad poisoning.

It is a further objective of the invention to provide a kit having one or more delivery units having pre-loaded amounts of toxin absorber solution for treatment of an animal exposed to toxic cane toad poisoning.

It is yet another objective of the invention to provide a method using one or more delivery units having pre-loaded amounts of toxin absorber solution for treatment of an animal exposed to toxic cane toad poisoning.

It is a further objective of the invention to provide a kit having one or more syringes having pre-loaded amounts of toxin absorber solution for treatment of an animal exposed to toxic cane toad poisoning.

It is yet another objective of the invention to provide a method using one or more syringes having pre-loaded amounts of toxin absorber solution for treatment of an animal exposed to toxic cane toad poisoning.

Other objectives and advantages of this invention will become apparent from the following description taken in conjunction with any accompanying drawings wherein are set forth, by way of illustration and example, certain embodiments of this invention. Any drawings contained herein constitute a part of this specification, include exemplary embodiments of the present invention, and illustrate various objects and features thereof.

### BRIEF DESCRIPTION OF THE FIGURES

FIG. 1 is an illustrative embodiment of a cane toad poisoning emergency response kit;

FIG. 2 is an illustrative embodiment of a water bottle;

FIG. 3 is an illustrative embodiment of a toxin absorber solution housed in a bottle;

FIG. 4 is an illustrative embodiment of a toxin absorber delivery device;

FIG. 5A is an illustrative embodiment of the syringe with a pre-loaded volume of the toxin absorber solution;

FIG. 5B is an illustrative embodiment of the syringe with a pre-loaded volume of the toxin absorber solution;

FIG. 5C is an illustrative embodiment of the syringe with a pre-loaded volume of the toxin absorber solution;

FIG. 6 is an illustrative embodiment of a pair of protective gloves;

FIG. 7 is an illustrative embodiment of protective glasses;

FIG. 8 is an illustrative embodiment of an absorbing material;

FIG. 9 is an illustrative embodiment of a portable flashlight;

FIG. 10 is an illustrative embodiment of a response guide booklet;

FIG. 11 illustrates the response guide booklet in an open position, illustrating toxin exposure treatment instructions;

FIG. 12 is an illustrative embodiment of a travel or carrying bag;

FIG. 13 is an illustrative embodiment of a breathing device for use in performing cardiopulmonary resuscitation (CPR) in non-humans;

FIG. 14 is an exploded view of the breathing device for use in performing cardiopulmonary resuscitation (CPR) in non-humans illustrated in FIG. 13;

FIG. 15A is a perspective view of an illustrative embodiment of an air delivery member;

FIG. 15B is an alternative perspective view of an illustrative embodiment of an air delivery member;

FIG. 16 is a cross-sectional view of the air delivery member, taken along lines A-A in FIG. 15B;

FIG. 17 is an illustrative embodiment of a barrier member, illustrated for use with small sized dogs/small snouts;

FIG. 18 is an illustrative embodiment of a barrier member, illustrated for use with medium sized dogs/medium snouts;

FIG. 19 is an illustrative embodiment of a barrier member, illustrated for use with large sized dogs/large snouts; and

FIG. 20 illustrates the breathing device for use in performing cardiopulmonary resuscitation (CPR) in non-humans engaging with and secured to a dog.

### DETAILED DESCRIPTION OF THE INVENTION

of While the present invention is susceptible embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

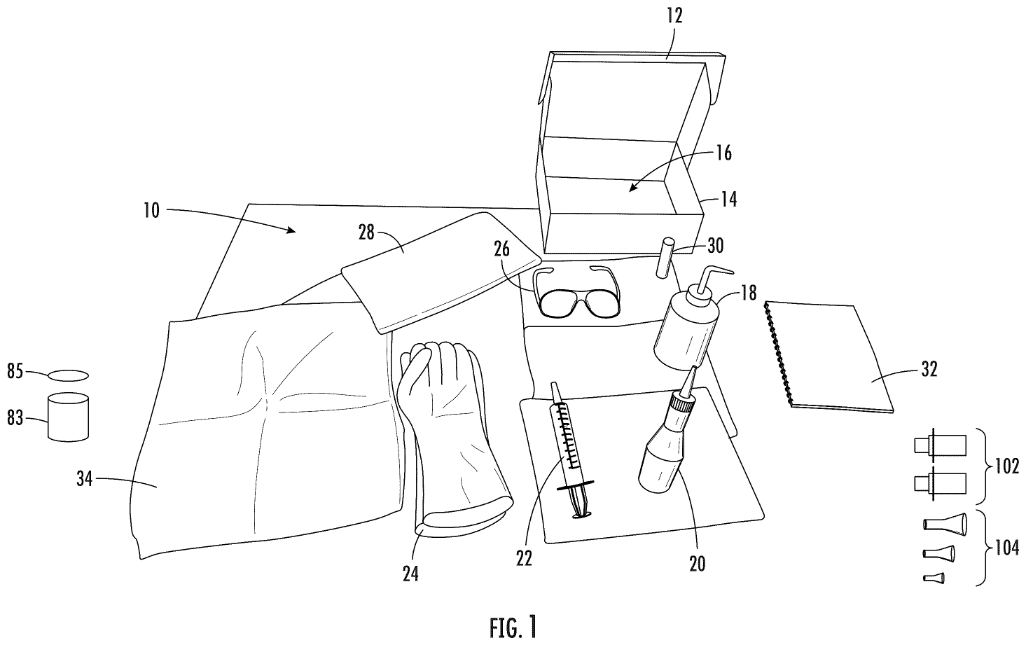

Referring to FIG. 1, an illustrative example of a cane toad poisoning emergency response kit, referred to generally as a cane toad poisoning kit 10, is shown. The cane toad poisoning kit 10 may include all of the components, or any combination of components. Each of the components that make up the cane toad poisoning kit 10 may be delivered and/or stored in a delivery unit 12, illustrated herein as a box 14 having an interior 16.

The delivery unit 12, while shown as a box, may take other forms, such as a backpack or sealable bag. The kit 10 may include a water bottle 18, a toxin absorber solution 20, a toxin absorber delivery device 22, a pair of protective gloves 24, safety glasses 26, one or more absorbing materials 28, a flashlight 30, a response guide 32, and a travel or carrying bag 34.

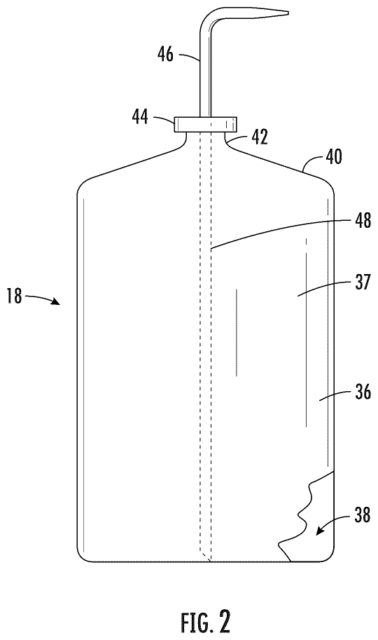

Referring to FIG. 2, an illustrative example of the water bottle 18 is shown. The water bottle 18 has a main body 36 having a continuous wall 37 enclosing an interior portion 38.

The interior portion 38 is designed to hold a liquid, such as water. An upper portion or end 40 includes a threaded neck 42 sized and shaped to receive a removable cap 44. The cap 44 includes a curved applicator tip 46, which is operatively connected to a tube 48 extending into the main body 36 interior portion 38. Preferably, the main body 36 is made of a squeezable material, such as a plastic material, so as to allow a user to apply pressure thereto, causing water to move from the interior portion 38 and exit out the curved applicator tip 46.

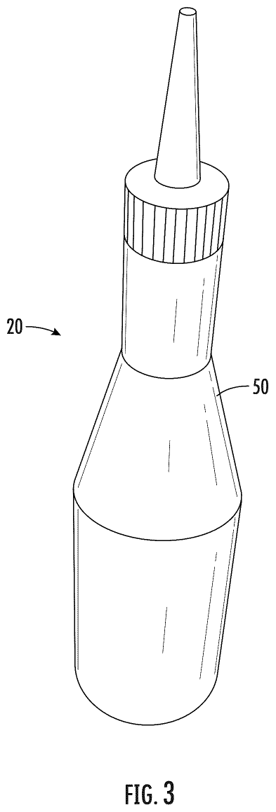

Referring to FIG. 3, an illustrative example of the toxin absorber solution 20, housed in a bottle 50, is provided. The toxin absorber solution 20 is illustrated herein as a Charcoal-Kaolin suspension. Alternatively, the toxin absorber solution 20 can be an activated charcoal gel which includes charcoal powder, Kaolin and Pectin.

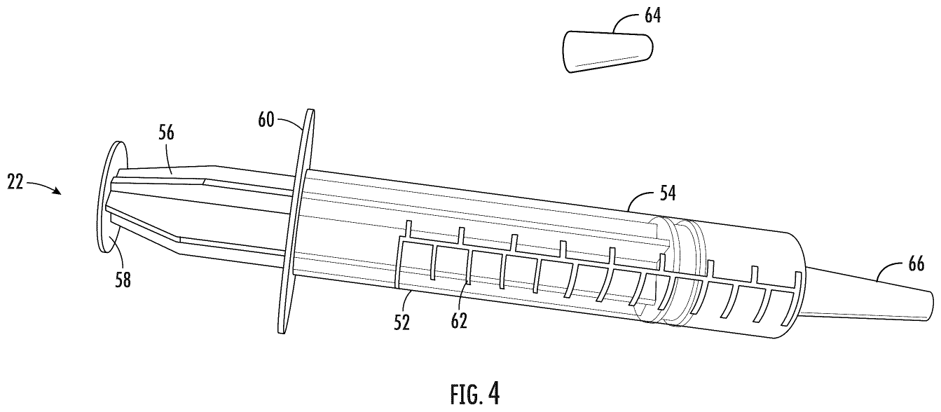

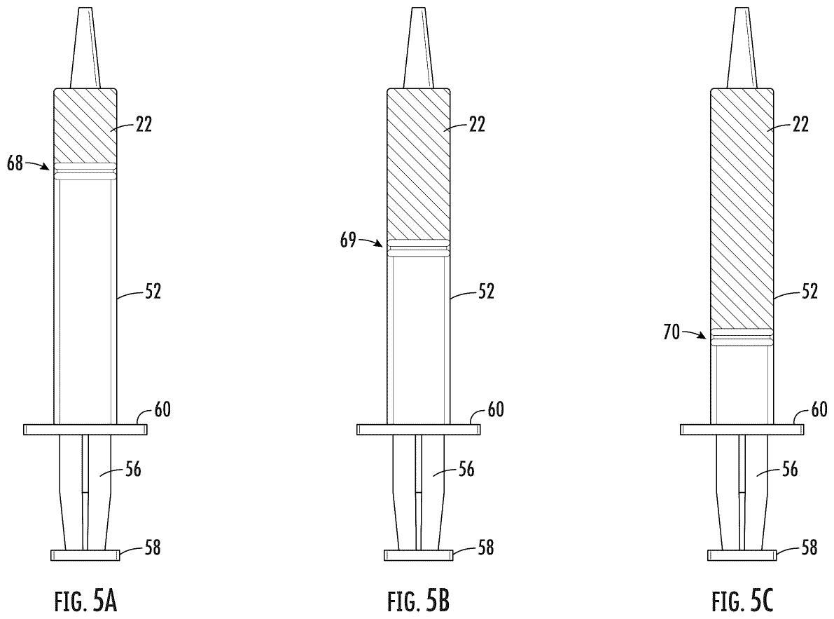

Referring to FIG. 4, an illustrative example of the toxin absorber delivery device 22 is provided. The toxin absorber delivery device 22 is provided as a syringe 52. The syringe 52 may include a barrel 54, a plunger 56, a thumb rest 58, and a finger flange 60. The barrel 54 may include a calibration scale 62. If needed, a tip protector 64 may be secured to the dispensing end 66. In use, a user may add the toxin absorber solution 20, from bottle 50, to the syringe 52. Alternatively, the syringe 52 may come pre-loaded with the toxin absorber solution; see FIGS. 5A-5C. The amount of the pre-loaded toxin absorber solution may be based on the weight of the animal. In FIG. 5A, the syringe 52 is shown with a 10 ml, see arrow 68, pre-loaded amount of toxin absorber solution 20. In

FIG. 5B, the syringe 52 is shown with a 25 ml, see arrow 69, pre-loaded amount of toxin absorber solution 20. In FIG. 5C, the syringe 52 is shown with a 50 ml, see arrow 70, pre-loaded amount of toxin absorber solution 20. Each cane toad poisoning kit 10 may include one or more pre-loaded syringes 52 of the same volume, or multiple pre-loaded syringes 52, each with a different volume.

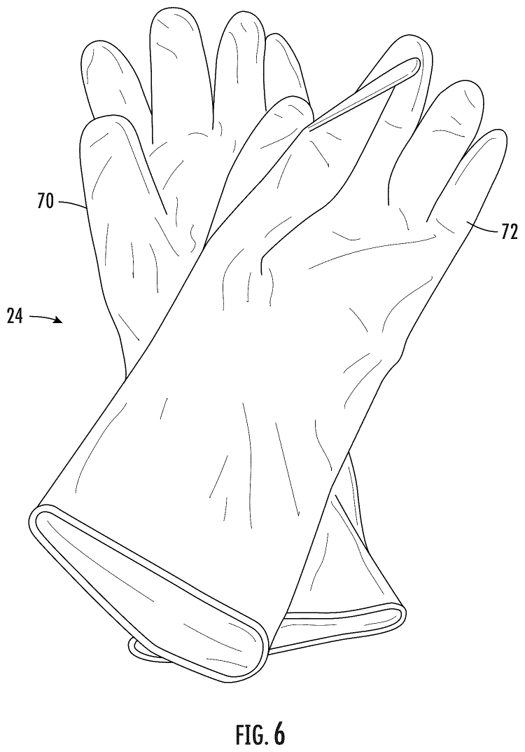

Referring to FIG. 6, an illustrative example of the pair of protective gloves 24 is shown. A glove for the user's left hand 70 and a glove for the user's right hand 72 are provided to protect the user from accidental exposure to the bufotoxin via, for example, splattering. The gloves 70, 72 can be made of any material that prevents exposure of toxins to the skin, and may include, for example, latex or nitrile.

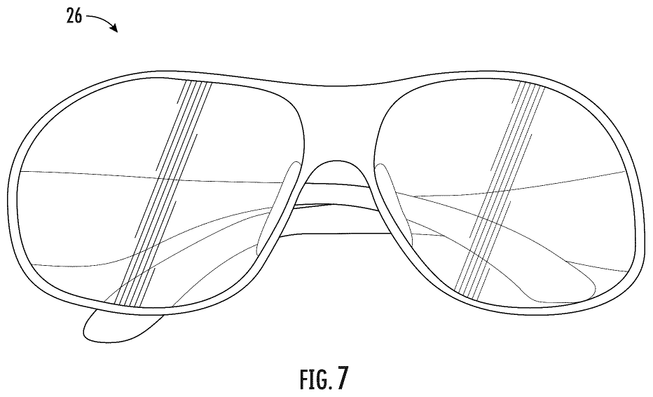

Referring to FIG. 7, an illustrative example of the safety glasses 26 are illustrated. The safety glasses 26 are constructed of a material that allows a user to see when wearing, but prevents exposure of the toxin to a user's eyes.

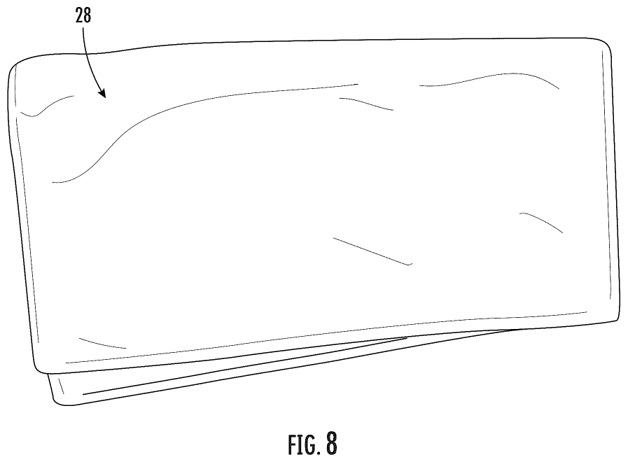

Referring to FIG. 8, an illustrative example of the one or more absorbing materials 28 is illustrated. The one or more absorbing materials 28 are illustrated as a towel made of an absorbent material. For ease of storage the towels may come disposable, and come in a compressed state. These towels are compressed into a small shape, such as a coin, and expand to a fuller size when opened.

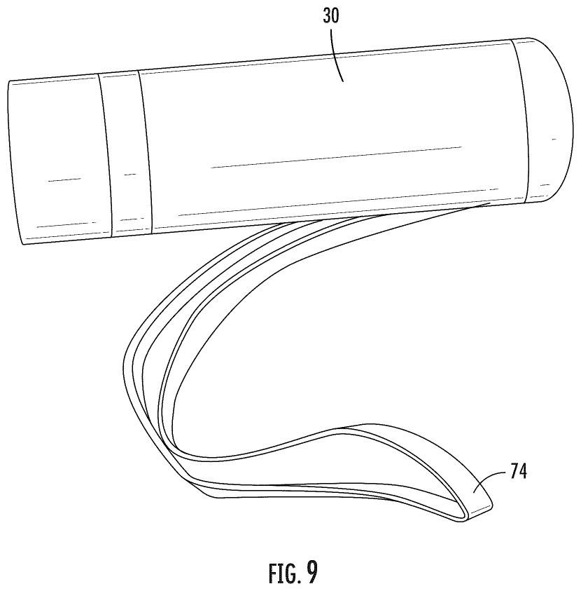

Referring to FIG. 9, an illustrative example of the flashlight 30 is shown. The flashlight 30 may include a carry strap 74.

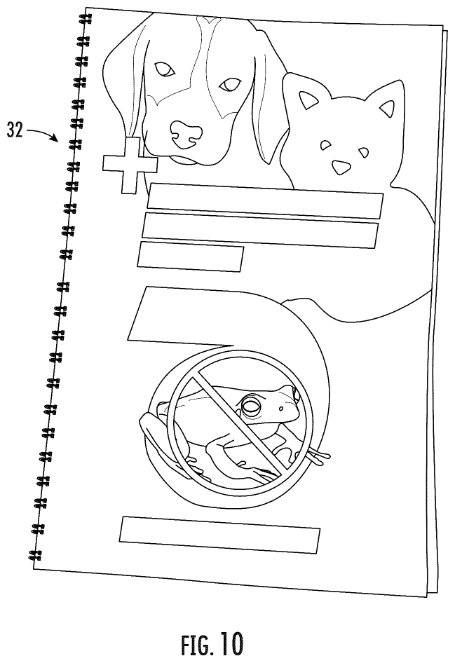

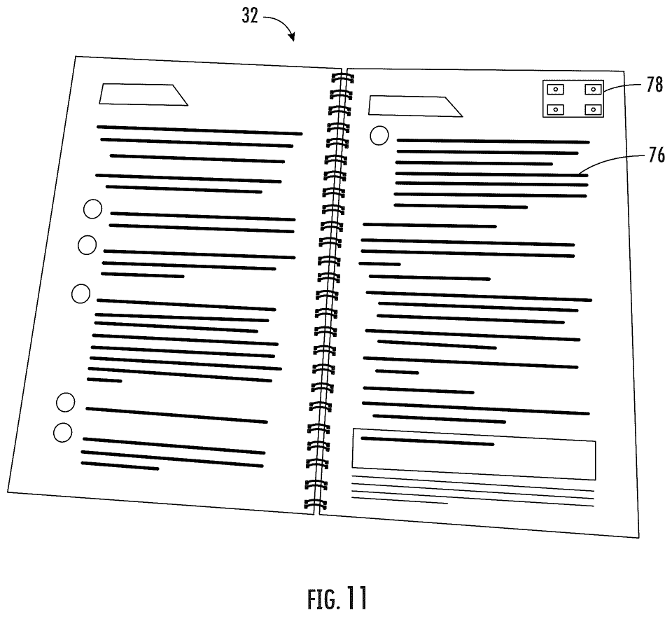

Referring to FIGS. 10 and 11, an illustrative example of the response guide 32 is shown. The response guide 32 may include written instructions 76, teaching a user how best to treat an animal that has been exposed to bufotoxin. The response guide 32 may also include a QR code 78. The QR code 78 is linked to a video illustrating how to treat an animal that has been exposed to bufotoxin. In addition, or alternatively, a QR code 78 may be placed on the delivery unit 12.

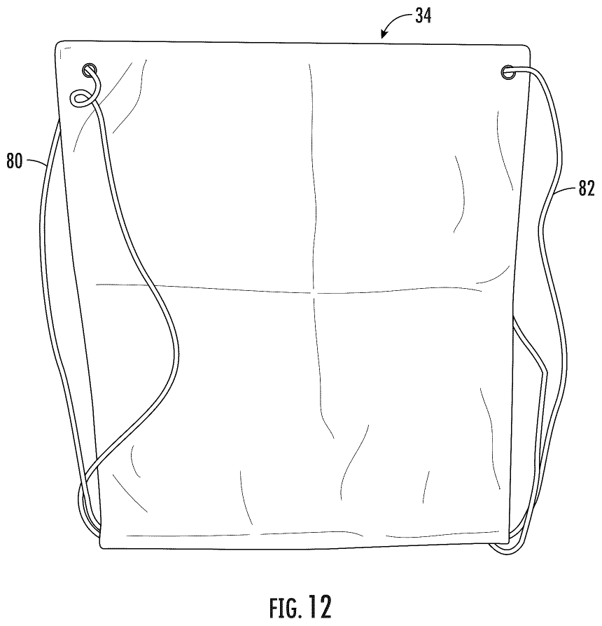

Referring to FIG. 12, an illustrative example of the travel or carrying bag 34 is shown. The travel or carrying bag 34 includes an interior in which one or more of the components of the cane toad poisoning kit 10 may be placed and stored therein. Straps or ropes 80, 82 may be used to allow a user to carry the travel or carrying bag 34 when away from home, such as on a nightly walk with their animal or on a camping trip. The straps or ropes 80, 82 may also be used as a means to close the bag tight, thus preventing the contents from spilling out.

In certain embodiments, the cane toad poisoning kit 10 may include one or more plastic bottles, 83, such as a plastic prescription vial or a pill bottle, which may be waterproof and airtight with a top cap or cover 85, such as a pop top cap or screw on cap/cover.

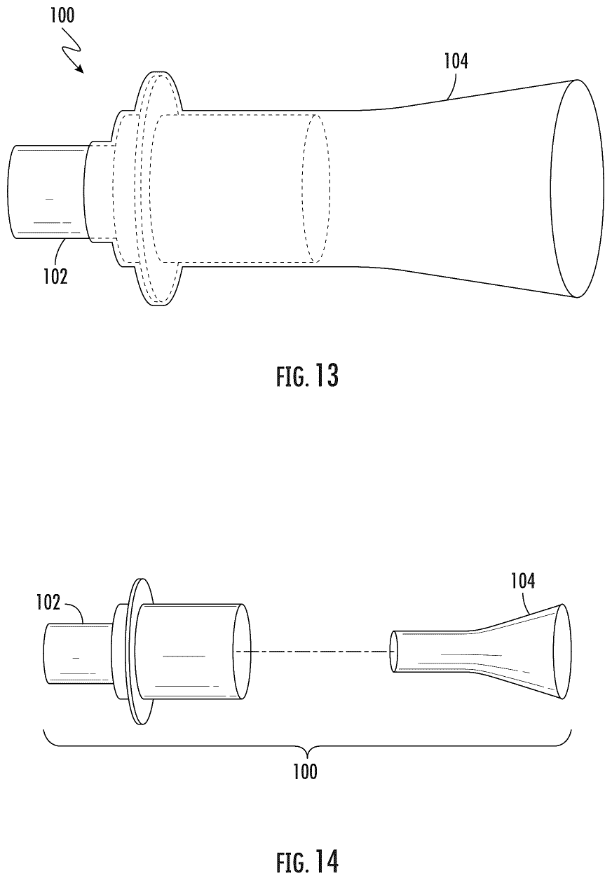

Referring to FIGS. 13-20, an illustrative embodiment of a breathing device for use in performing cardiopulmonary resuscitation (CPR) in non-humans, such as animals having a snout, such as canines, referred to generally as a CPR breathing apparatus for animals 100, is illustrated. The CPR breathing apparatus for animals 100 is designed to engage with or secure an animal, covering the animal's nose and/or mouth. For illustrative purposes, the CPR breathing apparatus for animals 100 will be described for use with a dog. However, the CPR breathing apparatus for animals 100 may be used with other animals as well.

Applying the CPR breathing apparatus for animals 100 to a dog in need thereof offers several benefits. First, the CPR breathing apparatus for animals 100 is designed to provide a more precise and proper fit on the dog. This ensures that the dog 1) receives the optimal amount of air per breath delivered to the dog during the rescue breaths portion of CPR, and 2) the air is more optimally received through the animal's nose. In addition, the CPR breathing apparatus for animals 100 is designed to provide a barrier between the dog and the human administering the rescue breaths. The barrier is designed to prevent the transfer of certain materials, substances, or fluids from the dog's mouth to the human's mouth during the CPR steps. In attempting to resuscitate a dog suffering from cane toad toxin poisoning, it is critical that the toxin does not transfer to the human's mouth during the CPR process.

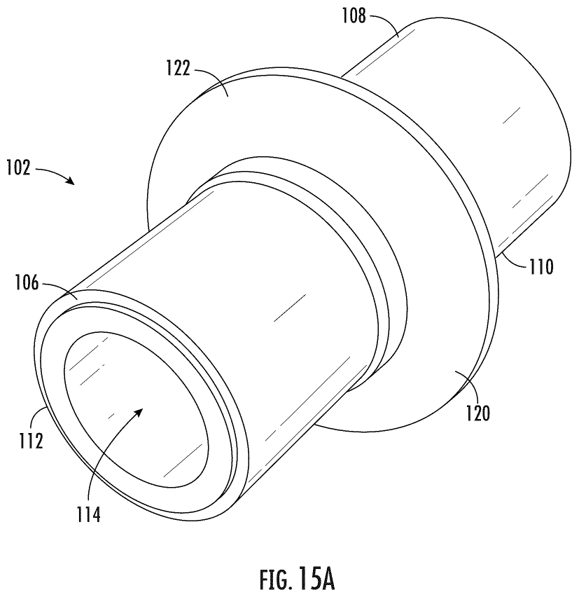

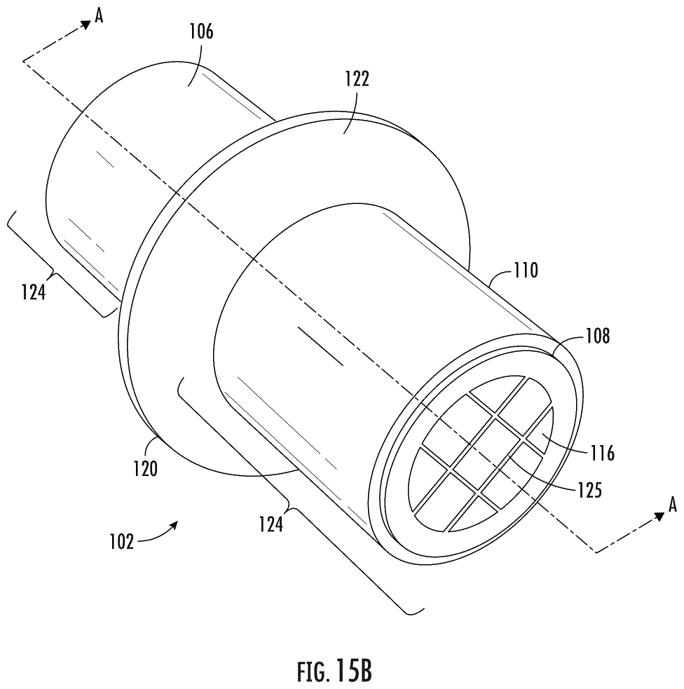

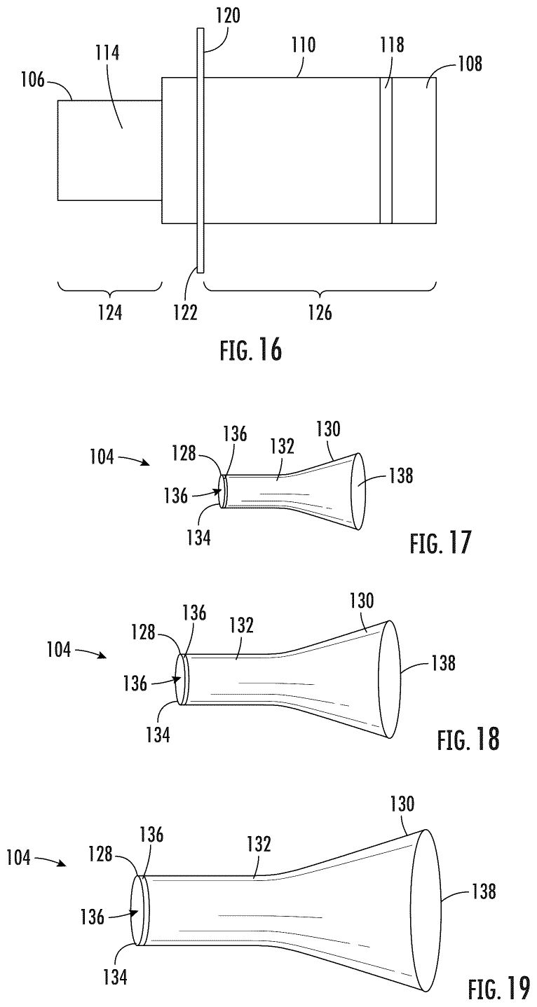

Referring specifically to FIGS. 13-14, the CPR breathing apparatus for animals 100 is shown having an air delivery member 102 and a barrier member 104. The air delivery member 102 is configured to allow for the movement of air from the human's mouth, into and through the barrier member 104, and into the dog's nose (or mouth). The air delivery member 102, illustrated herein as a two-way valve, see FIGS. 15A-16, comprises a first end 106, a second, opposing end 108, and a main body 110. The main body 110 separates the first end 106 and the second end 108. The first end 106 is configured to engage with and/or secure to an air producing source, such as a user's mouth. A first end opening 112 allows air to enter an interior portion 114 (forming a hollow or partially hollow lumen) of the main body 110. The second end 108 is configured to engage with and/or secure to a portion of the barrier member 104. A second end opening 116 is sized and shaped to allow air, traveling within the interior portion 114 to exit the air delivery member 102, and enter the barrier member 104.

A filter or membrane 118 may be used to prevent the movement or passage of materials or substances, such as the cane toad toxin, from back traveling, i.e, traveling from the animal (animal's mouth), back into or towards the air source, such as the human mouth. Preferably, the filter or membrane 118 is made of a material having a sufficient filtering capacity, such as a material having a sufficient pore size to prevent materials or substances from passing through the filer and traveling back towards the air source but allows for the movement of air towards the animal. The main body 110 may also include a stop member 120, illustrated herein as flanged body 122. The flanged body 122 extends outwardly and circumferentially about the main body 110. The flanged body 122 separates the main body 110 into a first portion 124 and a second portion 126. In certain embodiments, the first portion 124 has a smaller diameter than that of the second portion 126. When secured to the air delivery member 102, the stop member 120 prevents or minimizes the barrier member 104 from falling off the air delivery member 102. To prevent the filter from exiting out of the main body 110, the second end 108 may include a grate/grating 125.

Referring to FIGS. 17-19, embodiments of the barrier member 104 are shown in varied sizes (for small sized dogs/small snouts, FIG. 17; for medium sized dogs/medium snouts, FIG. 18; and for large sized dogs/large snouts, FIG. 19). The barrier member 104 is configured to engage with and/or secure to the dog's snout, covering the nostril openings and the nasal planum. The dog's mouth may be covered by the barrier member 104 as well. The barrier member 104 comprises a first end 128, a second, opposing end 130, and a main body 132 separating the first end 128 and the second end 130. The first end 128 is configured to engage with or secure to the air delivery member 102. A first end opening 134 allows air to enter an interior portion 136 of the main body main body 132. The second end 130 is configured to engage with or secure to a portion of the animal, i.e., the snout or the entire face. A second end opening 138 is sized and shaped to 1) allow air, traveling within the interior portion 136 to be delivered to the animal's snout, and 2) extend sufficiently to allow for engagement with and/or securing to the animal's snout.

The barrier member 104 is made of an elastic or flexible material that can stretch to cover the dog's snout when a force is applied to it, i.e., it is stretched by pulling it. When the force is removed, i.e., no longer being stretched, it returns to its original position or shape, or it holds its shape when positioned to cover the object it is stretched over, such as the dog's snout or the air delivery member 102. In an illustrative embodiment, barrier member 104 is made of non-latex rubber, such as nitrile. The first end 128 may include a thickened portion forming a ridge or rim 136 for added support.

The delivery member 102 and the barrier member 104 may form part of the cane toad poisoning kit 10. In such case, the delivery member 102 and the barrier member 104 may be included as separate components which would require a user to assemble them together. Alternatively, the delivery member 102 and the barrier member 104 may be supplied already assembled. The cane toad poisoning kit 10 may be provided so there are one or more, such as two, three, four etc., delivery members 102. The cane toad poisoning kit 10 may be provided so there are one or more, such as two, three, four etc., barrier members 104, where all the barrier members 104 are the same size (all smalls, all mediums, or all larges), or a mixture of smalls, mediums, and/or larges.

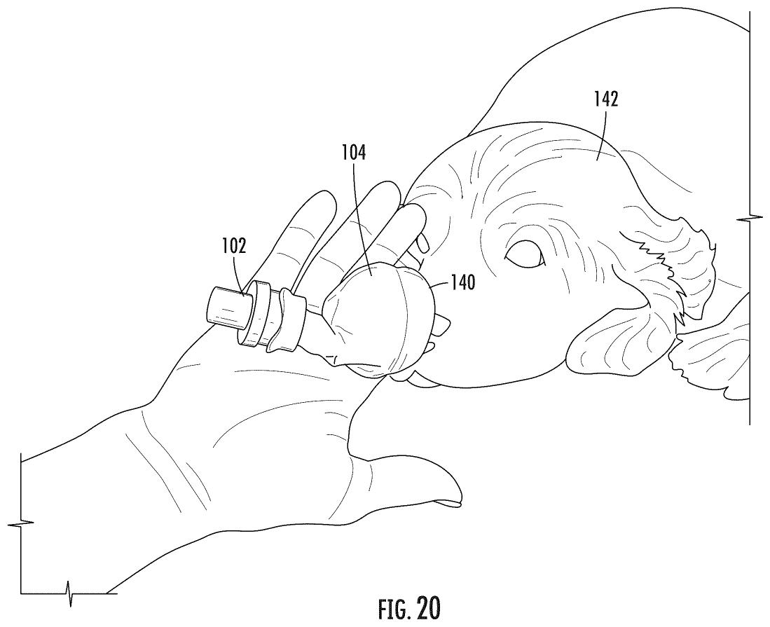

In use, when the dog requires CPR, such as when the dog is exposed to the cane toad toxin or poison and is unresponsive (not breathing), the user secures the air delivery member 102 to the barrier member 104 (placing the first end 128 of the barrier member 104 over the second portion 126 of delivery member 102). The second end 138 of the barrier member 104 is stretched and secured to a snout 140 of a dog 142, see FIG. 20. The barrier member 104 may be placed over the dog's snout, up to the corners of the dog's mouth. Air is provided to the dog by breathing through the first end opening 112 of the air delivery member 102. Additional breaths of air may be provided as required.

In use, the cane toad poisoning kit 10 provides an all in one, fast responsive device for initial treatment of an animal exposed to bufotoxin. The cane toad poisoning kit 10 not only provides for a quick response, but it also allows the user to be fully prepared for such an event. Precious treatment time is not wasted as a user searches for various items. A rapid response and having the necessary tools are critical for minimizing poor outcomes after bufotoxin exposure.

Upon exposure to the animal (illustrated as a dog, but applicable to other animals as well), a user should:
Step 1: First put on safety glasses 26 and gloves 24, 70, 72 to protect from harmful splatter.
Step 2: Using one of the one or more absorbing materials 28, a user can wipe the inside of the dog's mouth, including the tongue, cheeks, and palate.
Step 3: Using a the prefilled (with water) water bottle 18 with the curved applicator tip 46, the user rinses the dog's mouth, ensuring not to point the applicator tip 46 toward the back of the dog's mouth/throat. The user should attempt to rinse with the applicator tip 46 pointed toward the front of the mouth to avoid rinsing the bufotoxin toward the back where it may be swallowed by the dog. Alternatively, the user may use the water to saturate one of the one or more absorbing materials 28 and wipe the dog's mouth thoroughly.
Step 4: Repeat Steps 2 and 3 using a new one or more absorbing materials 28 with each wipe.
Step 5: After a final wipe, the user should administer the toxin absorber solution 20, i.e., the Charcoal-Kaolin suspension as described in Step 6.
Step 6: To use the Charcoal-Kaolin suspension, administer a volume, for example 1 ml per pound of the dog's body weight. For a 10-lb. dog, administration of the Charcoal-Kaolin suspension would be 10 ml. The Charcoal-Kaolin suspension is placed in the toxin absorber delivery device 22, i.e., the syringe 52, to the correct level/volume. The user may then push the plunger 56 to expel the Charcoal-Kaolin suspension out of the barrel 54 via the tip 66 directly into the dog's mouth. The plunger 56 should be pushed slowly enough to allow the dog to voluntarily swallow the Charcoal-Kaolin suspension.

If the kit comes with one or more pre-filled syringes 52, the user simply finds the syringe 52 that has the required amount of the Charcoal-Kaolin suspension and dispenses as above.

After the Charcoal-Kaolin suspension has been fully dispensed, the dog should be transported to a treating facility, such as a veterinary clinic or animal hospital for evaluation and/or further treatment.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures, and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A device for use in providing CPR to an animal comprising:
an air delivery member; and
a barrier member, said barrier member configured to secure to said air delivery member at a first end, and to an animal at a second end.

2. The device according to claim 1, wherein the air delivery member comprises a first end, a second end, and a main body separating the first end and the second end.

3. The device according to claim 1, wherein the barrier member is configured to engage with and/or secure to an animal's snout, said barrier member comprising a first end, a second end, and a main body separating the first end and the second end.

4. The device according to claim 1, wherein the barrier member first end comprises a first end opening, and the second end comprises a second end opening.

5. The device according to claim 1, wherein the barrier member is made of a flexible or stretchable material.

6. The device according to claim 5, wherein the flexible or stretchable material is nitrile.

7. The device according to claim 5, wherein the animal is a dog.

8. The device according to claim 1, wherein the air delivery member comprises a filter.

9. A kit for use in responding to toxic cane toad poisoning in animals comprising:
at least one apparatus for use in providing CPR to an animal, said apparatus comprising an air delivery member; and a barrier member, said barrier member configured to secure to said air delivery member at a first end, and to an animal at a second end.

10. The kit according to claim 9, further comprising one or more components that are usable for treating an animal suffering from toxic cane toad poisoning.

11. The kit according to claim 9, wherein the one or more components that are usable for treating an animal suffering from toxic cane toad poisoning comprises toxin absorber solution.

12. The kit according to claim 9, wherein the one or more components that are usable for treating an animal suffering from toxic cane toad poisoning comprises a toxin absorber delivery device.

13. The kit according to claim 9, wherein the one or more components that are usable for treating an animal suffering from toxic cane toad poisoning comprises a toxin absorber solution prefilled in a toxin absorber delivery device.

14. The kit according to claim 9, wherein the toxin absorber solution is activated charcoal.

15. The kit according to claim 9, wherein the one or more components that are usable for treating an animal suffering from toxic cane toad poisoning comprises a bottle for holding and delivering a fluid.

16. The kit according to claim 9, wherein the one or more components that are usable for treating an animal suffering from toxic cane toad poisoning comprises protective gear.

17. The kit according to claim 9, wherein the protective gear includes protective gloves or safety glasses.

18. The kit according to claim 9, including two or more barrier members.

19. The kit according to claim 18, wherein the two or more barrier members are the same size.

20. The kit according to claim 18, wherein the two or more barrier members are varied sizes.
